(19) United States
(12) Patent Application Publication
(10) Pub. No.: US 2022/0378605 A1
(43) Pub. Date: Dec. 1, 2022

(54) SMART HEAD COOLING SYSTEM

(71) Applicant: AYYA, Inc., Mission Viejo, CA (US)

(72) Inventors: Sawsan (Susan) Abushakra, Mission Viejo, CA (US); Michael A. Jarpe, Mission Viejo, CA (US); Orlando P. Rodrigues, Mission Viejo, CA (US)

(21) Appl. No.: 17/818,198

(22) Filed: Aug. 8, 2022

Related U.S. Application Data
(63) Continuation of application No. PCT/US2021/016780, filed on Feb. 5, 2021.
(60) Provisional application No. 62/971,092, filed on Feb. 6, 2020.

(51) Int. Cl. A61F 7/00; A42B 1/008; A42B 1/017

(52) U.S. Cl. CPC A61F 7/007; A42B 1/008; A42B 1/017; A61F 2007/0075; A61F 2007/0064; A61F 2007/0008; A61F 2007/0078; A61F 2007/0094

(57) ABSTRACT

A headpiece includes a shell, a thermoelectric cooler, a heat sink and a fan. The shell defines a concavity. The thermoelectric cooler includes a cooling surface and a warming surface. The heat sink is in thermal communication with the warming surface. The fan is in fluid communication with the heat sink.

### REFERENCE TO RELATED APPLICATION

This application is a continuation application that claims priority to International Patent Application No. PCT/US2021/016780, filed Feb. 5, 2021 now publication no. WO 2021/158893A1, which claims priority to U.S. Provisional Application No. 62/971,092, filed Feb. 6, 2020, which are incorporated by reference herein in their entireties.

### BACKGROUND

Migraines are characterized by moderate to severe headaches that are typically pulsating or throbbing in nature. A migraine headache may typically be unilateral. That is, a migraine may typically manifest as a headache affecting a left side or a right side of a person's head. Other potential symptoms of a migraine may include nausea, vomiting, and sensitivity to light, sound, smell, and touch.

By some accounts, migraine headaches are the third most prevalent illness in the world and the sixth leading cause of disability. The severity of migraine headaches and the symptoms associated with migraine headaches may be disabling in a majority of those who experience migraines. A person experiencing a migraine may often require bedrest. Acute migraine episodes may occur with a frequency of 1-2 per month but may occur more frequently, and may be related to onset of menses (menstrual migraine). Untreated migraine episodes may generally last from a few hours to a few days. Those who experience 14 or more migraine days per month may be classified as experiencing chronic migraines. Those who frequently experience migraines may have their social, work, and leisure activities impacted. Migraines may also be associated with social withdrawal, depression, lost work time, and decreased productivity.

The claimed subject matter is not limited to embodiments that solve any disadvantages or that operate only in environments such as those described above. This background is only provided to illustrate examples of where the present disclosure may be utilized.

### SUMMARY

One general aspect includes a headpiece including a shell defining a concavity, a thermoelectric cooler having a cooling surface and a warming surface, a heat sink in thermal communication with the warming surface, and a fan in fluid communication with the heat sink. The headpiece may include a plurality of thermoelectric coolers having a plurality of cooling surfaces and a plurality of warming surfaces. The heat sink may be in thermal communication with each warming surface of the plurality of warming surfaces. In some aspects, the headpiece may include a plurality of heat sinks, each heat sink of the plurality of heat sinks may be in thermal communication with at least one warming surface of the plurality of warming surfaces and each fan of the plurality of fans may be in fluid communication with at least one heat sink of the plurality of heat sinks. In some aspects, the shell may include: a first cap portion; a second cap portion; and a bridge portion connected to the first cap portion and the second cap portion. The bridge portion may be connected to the first cap portion by a first hinge. The bridge portion may be connected to the second cap portion by a second hinge. The fan may be located on the bridge portion. The first cap portion may include a first body and a first cover forming a first housing; the heat sink may include a plurality of thermally conductive fins located within an interior of the first housing; and the fan may be in fluid communication with the interior of the first housing. The second cap portion may include a second body and a second cover forming a second housing; a battery and a controller of the headpiece may be located within the second housing. The headpiece may include a flexible fluid conduit forming a fluid connection between an intake opening of the fan and the interior of the first housing; the first cover may form an opening adjacent to the thermally conductive fins of the heat sink.

One general aspect includes a system including the headpiece and a mobile device configured to execute an application configured to remotely control operation of the headpiece. In some aspects, the application may be configured to collect and store data associated with at least one of characteristics of a migraine experienced by a user of the headpiece, symptoms of the migraine, and triggering factors associated with the migraine.

### BRIEF DESCRIPTION OF THE DRAWINGS

FIG. 1 illustrates an example environment.

FIG. 2A illustrates a perspective view of an example headpiece.

FIG. 2B illustrates a top view of the example headpiece of FIG. 2A.

FIG. 3A illustrates a top perspective view of an example cooling element.

FIG. 3B illustrates a bottom perspective view of the example cooling element of FIG. 3A.

FIG. 4A illustrates a top perspective view of another example cooling element.

FIG. 4B illustrates a bottom perspective view of the example cooling element of FIG. 4A.

FIG. 5A illustrates a perspective view of another example headpiece.

FIG. 5B illustrates another perspective view of the example headpiece of FIG. 5A.

FIG. 5C illustrates the example headpiece of FIG. 5A with a cover omitted.

FIG. 5D illustrates the example headpiece of FIG. 5B with a cover omitted.

FIG. 5E illustrates a front view of the headpiece of FIGS. 5C and 5D.

### DETAILED DESCRIPTION

Migraines may be associated with symptoms of autonomic instability, where a person experiencing a migraine may experience unpleasant alternating sensations of heat and of cold, as well as fluctuations in blood pressure. Alternately or additionally, migraines may be associated with symptoms of allodynia, or increased sensitivity to touch and cold, the severity of which may increase with the severity of the migraine. Those who experience allodynia as an associated symptom of a migraine may experience the allodynia in a number of ways. For instance, allodynia may affect the skin unilaterally on the same side of the body as is experiencing the migraine or may be experienced bilaterally. Furthermore, allodynia may be experienced on the head, but not the body, or may be experienced on the head as well as the body.

Migraine treatment may include bedrest, over-the-counter analgesics and non-steroidal anti-inflammatory (NSAID) medications. Prescription medications and treatments may include over-the-counter mixed analgesics (such as acetaminophen combined with aspirin and caffeine, or acetaminophen mixed with decongestants), ergots, triptans, calcitonin gene-related peptide (CGRP) antagonist injections, opioids, anti-epileptic psychotropic drugs, and botulinum toxin injections. These medications may be associated with adverse or unpleasant side effects. The use of opioids, for instance, may be associated with risk of overdose and addiction. Furthermore, aspirin and NSAIDs may cause gastric distress, triptans may cause chest tightness and increased blood pressure, and high doses of acetaminophen may cause liver toxicity. Ingesting oral medications may also be problematic for people experiencing nausea and vomiting. Additionally, many people who experience migraines may avoid injectable medications due to needle phobias or because arranging to receive the injection is not practical or feasible at the time the migraine is experienced.

Alternatives and/or adjuncts to pharmacological treatments for migraines may include head cooling. For instance, cold compresses, such as ice placed in a bag, gel packs that require freezing, or the like may be placed against the head of the person on the side of the head experiencing migraine pain. Cooling caps may also be used to provide head cooling. Conventional cooling caps may generally rely on the cap being kept in a freezer or the like for a time to cool a gel within the cap. Alternatively, ice or other cold inserts may be cooled separately and then inserted into designated compartments of the caps. Cooling caps may decrease headache severity and, potentially, uncomfortable sensations of heat that may be associated with migraines frequently experienced by some people.

The use of cold compresses and cooling caps may be inconvenient for many who experience migraines. For instance, in the case of gel packs, the gel packs need to be suitably cooled before they may be used effectively. Thus, gel packs that have warmed in the process of providing relief to a person experiencing a migraine will typically need to be re-cooled for a time. This potentially leaves the person experiencing the migraine without the relief provided by the gel pack. Even if multiple gel packs or ice are used to ensure more consistent cooling, the act of replacing the gel packs or ice may be particularly inconvenient or painful for a person experiencing a disabling migraine, since movement and light usually exacerbate the pain.

Furthermore, conventional cooling caps may cover and cool both sides of a head of a user. For those who experience allodynia, cooling the side of head contralateral to the side of the head experiencing the migraine may cause discomfort due to cold sensitivity.

Some embodiments of the systems detailed herein may facilitate unilateral cooling of one side of the head of a user. Accordingly, for example, a user experiencing a migraine and allodynia may unilaterally cool the side of the head ipsilateral to the migraine. This may provide the user some degree of relief from the migraine without promoting discomfort by cooling the side of the head contralateral to the migraine. Alternately, the systems may facilitate cooling of the head of a user without ice or gels that require cooling. In some embodiments, the systems may include a headpiece, which may include external electric coolers to cool water, which is then circulated to the head of a user wearing the headpiece.

Alternately or additionally, the systems may include an application run on a mobile device that may remotely control the functions of the headpiece. In some embodiments, the application may track usage of the headpiece such that the application may assist in tracking, for example, one or more of an intensity, timing, frequency, or duration of migraines experienced by a user.

Treating migraines, at least in part, through non-pharmacologic means may reduce the risk of side effects associated with pharmacologic migraine treatments by diminishing the user's reliance on those treatments. Those who experience migraines and use concomitant medications to treat other medical conditions may further reduce the risk of drug-drug interactions.

Reference will now be made to the figures wherein like structures will be provided with like reference designations. The drawings are non-limiting, diagrammatic, and schematic representations of example embodiments, and are not necessarily drawn to scale.

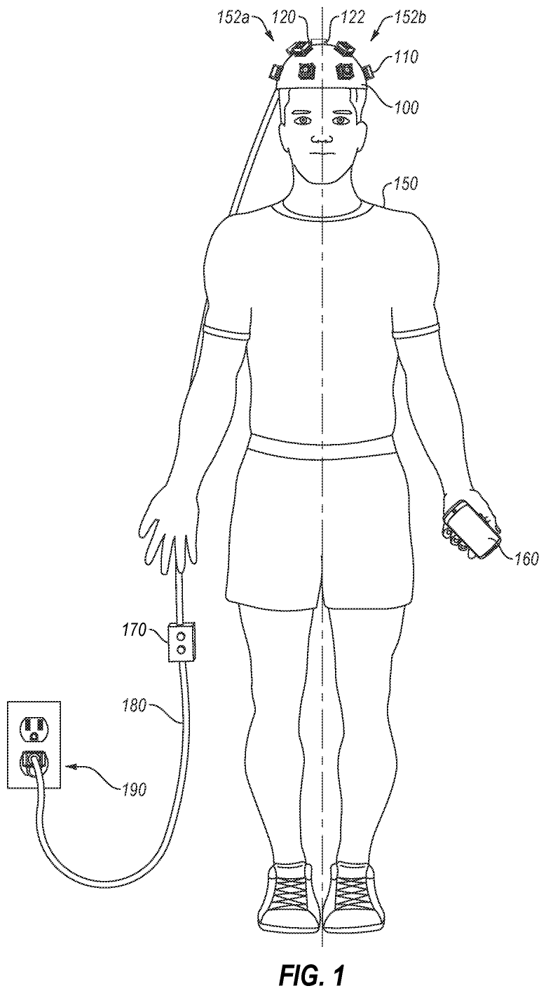

FIG. 1 illustrates an example environment in which some embodiments may be employed. In some embodiments, an example headpiece 100 may be configured to be worn on the head of a user 150. The headpiece 100 may be adjustable such that the headpiece 100 may be worn comfortably by the user 150. Adjustments may be accomplished via hook and loop fasteners, friction-held straps, buckles, clips, snap fasteners, or the like.

In some embodiments, functions of the headpiece 100 may be controlled via a wireless controller 160. Optionally, the headpiece 100 may be alternately or additionally controlled via an integrated controller 170. The integrated controller 170 may be located on a power supply line 180, which may deliver power to the headpiece 100 from a power source 190. Alternately or additionally, the integrated controller 170 may be located on the headpiece 100. For instance, in some embodiments, the headpiece 100 may be operated via battery power and may omit the power supply line 180. Alternatively, the power supply line 180 may recharge a battery 120 and may be removed when the battery 120 is not being charged.

The wireless controller 160 or the integrated controller 170 may allow the user 150 to operate the headpiece 100. For instance, the wireless controller 160 or the integrated controller 170 may instruct the headpiece 100 to cool the head of the user. The headpiece 100 may unilaterally cool a right side 152a or a left side 152b of the head of the user 150. For example, instructing the headpiece 100 to cool the right side 152a of the head of the user 150 may cause the right side 152a of the headpiece 100 to perform a cooling operation while the left side 152b of the headpiece 100 does not perform a cooling operation. Thus, for example, the headpiece 100 may provide cooling to a side of the head of the user 150 that is experiencing migraine pain without providing cooling to the other side of the head of the user 150, which may be experiencing allodynia associated with the migraine. In some embodiments, the headpiece 100 may perform cooling operations on both the right side 152a and the left side 152b of the headpiece 100. By way of example, the user 150 may desire to cool both the right side 152a and the left side 152b of the head of the user 150 at the same time when the user 150 is experiencing a migraine without accompanying allodynia.

The wireless controller 160 or the integrated controller 170 may further facilitate selection of an intensity of the cooling operation. In some embodiments, the wireless controller 160 or the integrated controller 170 may facilitate selection of an intensity of the cooling operation via an intensity selection sliding scale, which by way of illustration, may allow the user 150 to dial the intensity to some fraction of maximum intensity. Alternatively, the wireless controller 160 or the integrated controller 170 may facilitate selection of an intensity from a number of pre-defined intensity settings, such as low, moderate, or high cooling intensities.

The headpiece 100 may cool the scalp, forehead, temple, and thereabout of a selected right side 152a or left side 152b of the head of the user 150 by 1-4 degrees centigrade. In some configurations, the headpiece 100 may cool a selected right side 152a or left side 152b more than 4 degrees centigrade, although excessive cooling may risk injury to the skin of the user 150. The headpiece 100 may also cool the scalp on both sides if appropriate or needed.

In some embodiments, the headpiece 100 may be powered by the battery 120. The battery 120 may be rechargeable, either while the headpiece 100 is in use or while the headpiece 100 is not in use. For instance, the battery 120 may be recharged by way of a power supply line 180 that may be connected to a power source 190. In some configurations, the power supply line 180 may be a universal serial bus (USB) connector that, where needed, may be connected to a power source 190 by the way of an adapter. The wireless controller 160 or the integrated controller 170 may communicate a state of charge of the battery 120 such that it may be monitored by the user 150. Optionally, the wireless controller 160 or the integrated controller 170 may communicate an estimated operation time of the headpiece 100 given the state of charge of the battery 120 and the rate of battery charge depletion associated with the settings of the headpiece 100 at the time. Alternately or additionally, the wireless controller 160 or the integrated controller 170 may provide an alert when the battery 120 has reached a particular state of charge or a particular estimated operation time remaining. For instance, the wireless controller 160 or the integrated controller 170 may provide an alert in response to the state of charge of the battery 120 reaching 10% or less or in response to the battery 120 reaching a state of charge estimated to provide 5 minutes or less of operation time. By way of example, such alerts may notify the user 150 to expect to begin the battery 120 recharging process.

In some embodiments, the headpiece 100 may operate by way of power supplied through the power supply line 180 from the power source 190. For instance, the headpiece 100 may prioritize operations over battery 120 recharging when the headpiece 100 is connected to a power source 190 and instructed to perform cooling operations through the right side 152a or left side 152b of the headpiece 100. As a result, a user 150 experiencing a migraine may continue to employ the headpiece 100 to provide relief even while the battery 120 is being charged following depletion of the battery 120. Alternatively, the headpiece 100 may prevent operation while power is supplied through the power supply line 180 from the power source 190. In such configurations, the user 150 may be encouraged to cease operations of the headpiece 100 while the battery 120 charges, which may diminish a risk of skin damage through prolonged durations of cooling.

In some embodiments, the wireless controller 160 or integrated controller 170 may facilitate selection of a duration of the cooling operation. For example, the wireless controller 160 or the integrated controller 170 may facilitate input of a particular length of time the cooling operation is to run, such as 5, 10, or 15 minutes, although other lengths of time may be input. Optionally, the wireless controller 160 or the integrated controller 170 may impose a maximum limit to the length of time that may be input. For instance, the wireless controller 160 or the integrated controller 170 may limit the length of time that may be input to 60 minutes, 90 minutes, or 120 minutes, although other lengths of time may be selected as a limit. Limiting the length of time that the cooling operation may be run may act as a safety feature to discourage skin damage through prolonged lengths of cooling.

In some embodiments, the headpiece 100 may include a control module 122. The control module 122 may control the cooling operations of the headpiece 100. For instance, the control module 122 may control the cooling operations of the headpiece 100 consistent with the input received via the wireless controller 160 or the integrated controller 170. In some configurations, the control module 122 may execute instructions received from the wireless controller 160 or the integrated controller 170 for an instructed duration without additional instructions from the wireless controller 160 or the controller 170.

The headpiece 100 may perform cooling operations by way of cooling elements 110. The cooling elements 110 may include thermoelectric coolers (TECs), which may also be described as Peltier coolers. Alternately or additionally, the cooling elements 110 may include coolers that are not Peltier coolers. The cooling elements 110 may be operated to perform the cooling operation. For instance, the control module 122 may control power supplied to the cooling elements 110, such that the cooling elements 110 perform cooling consistent with the instructions received from the wireless controller 160 or the integrated controller 170.

In some embodiments, the headpiece 100 may cycle the operations of the cooling elements 110 to discourage skin damage resulting from prolonged cooling.

Furthermore, cycling the operation of the cooling elements 110 may decrease a rate at which the battery 120 is drained of its charge.

In some configurations, on durations and off durations of a cycle may be equal. For example, the cooling elements 110, consistent with the instructed cooling operation, may be supplied power for 5 minutes such that the cooling elements 110 provide cooling for 5 minutes, then may be denied power for 5 minutes. This cycle may be repeated until the instructed duration is achieved. In some configurations, an unequal cycle may be used. For example, a 10 minute on and a 5 minute off cycle may be repeated until an instructed duration is achieved.

In some embodiments, the user 150 may define the cycle durations via the wireless controller 160 or the integrated controller 170. In some configurations, the cycle lengths may be restricted to a predefined range to discourage skin damage from overcooling. For example, one or both of a maximum on duration and a minimum off duration may be defined. Alternatively, the cycle lengths may be pre-defined and the user 150 may not alter the cycle lengths. For instance, the cooling elements 110 may operate with on-off cycles of preset lengths. Alternatively, the cycle lengths may have default lengths that may be changed via the wireless controller 160 or the integrated controller 170.

In some configurations, the on duration of the cycle may be equal to or between 5 minutes and 10 minutes. Alternately or additionally, the off duration of the cycle may be equal to or between 5 minutes and 10 minutes. Alternately or additionally, the on and off durations of the cycle may be less than 5 minutes or more than 10 minutes.

In some embodiments, the wireless controller 160 may be a part of an application that runs on a mobile device such as a personal mobile phone associated with the user 150. The wireless controller 160 may further track use of the headpiece 100. For example, the wireless controller 160 may track which days, as well as the times of those days, that the headpiece 100 was employed. Alternately or additionally, the wireless controller 160 may track the duration the headpiece 100 was used, the intensity of the cooling, the side or sides of the head cooled, and the like.

Alternately or additionally, the wireless controller 160 may perform migraine trigger tracking. For example, the wireless controller 160 may perform tracking of triggers that may be associated with an increased risk of triggering or contributing to triggering a migraine. Some triggers may be tracked automatically, such as weather, barometric pressure, hours of sleep when paired with a sleep tracking device or software, relation to menses in users who experience menstrual migraines, and the like. Some triggers may be tracked manually, such as high stress, jet lag, intense exercise, and other lifestyle triggers, such as consumption of wine, cheese, chocolate, and other food triggers.

Headpiece 100 usage and trigger tracking may allow the user 150 to form a record of potential triggers, as well as a number of migraines experienced, the days and times the migraines were experienced, a duration and intensity of the migraine, whether allodynia was experienced, and the like. Such information and potentially the concomitant use of artificial intelligence (AI) to aid predictability may be shared with a physician to improve treatment plans. In some configurations, the wireless controller 160 may deliver migraine news and tips to the user 150, including info on new migraine treatments.

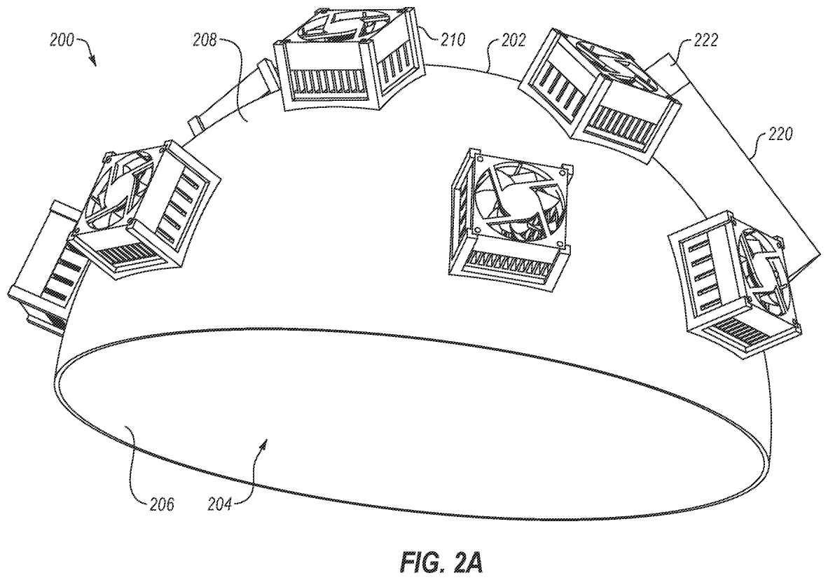

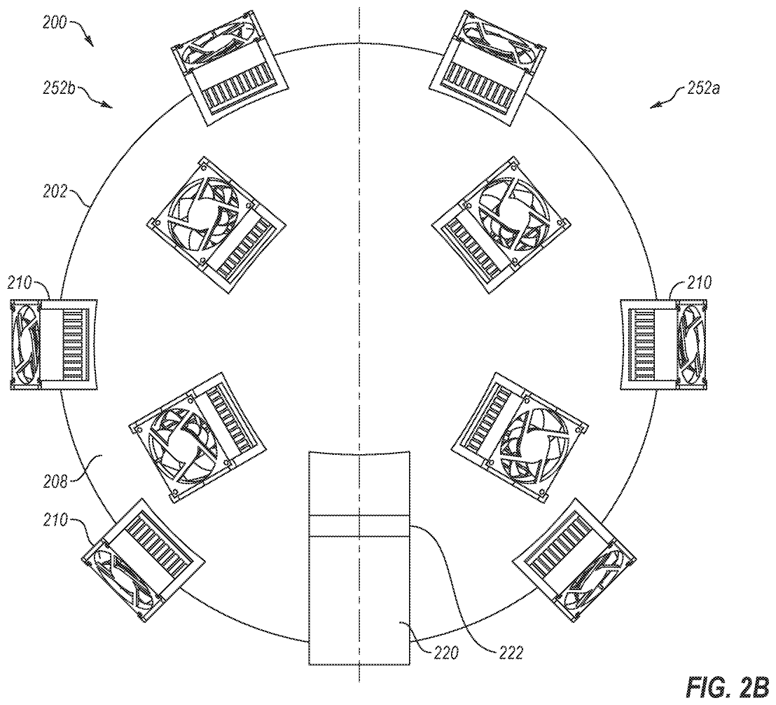

FIGS. 2A and 2B illustrate, respectively, a perspective view and a top view of an example headpiece 200, which may generally correspond to the headpiece 100 of FIG. 1. The headpiece 200 may include cooling elements 210, a battery 220, a control module 222, a right side 252a, and a left side 252b, which may generally correspond, respectively, to the cooling elements 110, the battery 120, the control module 122, the right side 152a, and the side 152b of FIG. 1.

The headpiece 200 may include a shell 202 shaped to form a concavity 204 such that the headpiece 200 may be positioned on a head of a user, such as the user 150 of FIG. 1. The cooling elements 210 may be affixed to the shell 202 such that a cooling surface of the cooling elements 210 faces the concavity 204 of the shell 202. For example, the cooling elements 210 may be affixed to the shell 202 such that the cooling surface of the cooling elements 210 is directed toward the concavity 204 and toward a head of a user wearing the headpiece 200.

The shell 202 may generally be flexible such that the shell 202 may fit a variety of head shapes. The shell 202 may further include adjustment features such that a size of the concavity 204 of the shell 202 may be made relatively larger or relatively smaller such that a user may fine tune a size of the headpiece 200 to fit the user comfortably.

The shell 202 may include a first material 206 that contacts skin or hair of the user when worn. In some embodiments, the first material 206 may include a thermally conductive material. In some configurations, the first material 206 may be positioned over cooling surfaces of the cooling elements 210 such that the cooling elements 210 does not contact the user directly. Alternately, the first material 206 may define openings sized and positioned such that the cooling surfaces of the cooling elements 210 may contact the user directly.

The headpiece 200 is shown with 5 cooling elements 210 per side of the headpiece 200. Alternately, more or fewer cooling elements 210 may be used. For example, each side of the headpiece 200 may include 2, 3, 4, 5, 6, 7, 8, 9, 10, 11, 12, 13, 14, or 15 cooling elements 210. In some embodiments, the cooling elements 210 may be positioned such that the cooling elements 210 are relatively evenly spaced. The cooling elements 210 may be attached to a second material 208 of the shell 202. The second material 208 may include neoprene, although the second material 208 may alternately or additionally include other materials.

In some configurations, the cooling elements 210 may be affixed to one side of the shell 202 such that the cooling elements 210 are located on only one side of the headpiece 200. For instance, in some embodiments, the cooling elements may be located only on the right side 252a of the headpiece 200. In such configurations, a user may selectively rotate the headpiece 200 as needed to selectively cool the right side or the left side of the user's head.

In some embodiments, electrically conductive lines and connectors between the cooling elements 210, battery 220, control module 222, and other electronics may be located between the first material 206 and the second material 208.

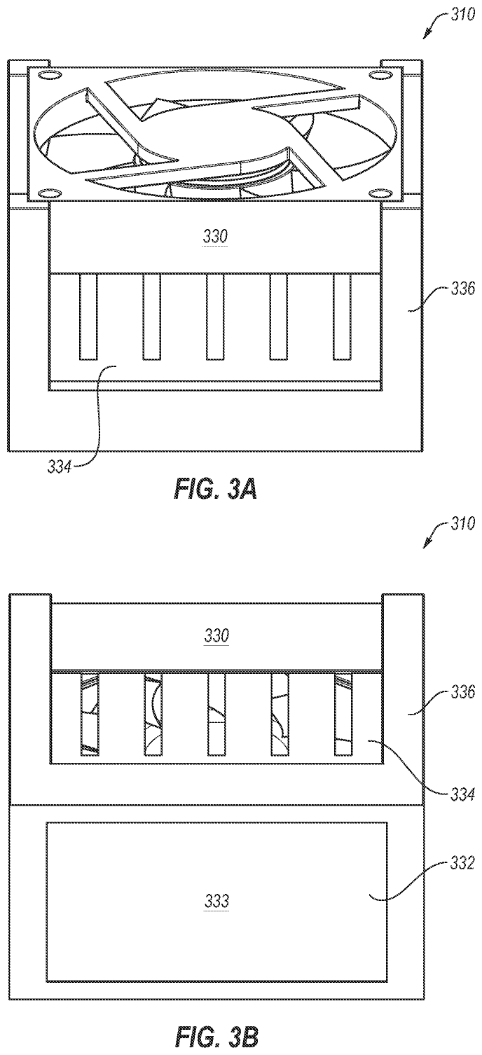

FIGS. 3A and 3B illustrate, respectively, top perspective and bottom perspective views of an example cooling element 310. Electrically conductive lines that supply power to the cooling element 310 are omitted for clarity. The cooling element 310 may generally correspond to the cooling elements 110 of FIG. 1 and the cooling elements 210 of FIGS. 2A and 2B. The cooling element 310 may include a TEC 332. In operation, the TEC 332 may cause a cooling surface 333 to become colder than an ambient temperature. In response, a warming surface located opposite the cooling surface 333 of the TEC 332 may become warmer than the ambient temperature. The heat generated at the warming side is described herein as waste heat.

A heat sink 334 may be positioned against the warming surface to promote conduction of heat away from the warming surface. In some configurations, a thermally conductive material such as thermally conductive paste may be located between the warming surface and the heat sink 334 to further promote heat transfer from the warming surface of the TEC 332 and the heat sink 334.

In some embodiments, the cooling element 310 may include a fan 330. The fan 330 may be in fluid communication with the heat sink 334 such that during operation of the cooling element 310, the fan 330 may promote airflow across the heat sink 334, which may promote heat transfer from the heat sink 334 to surrounding air via convection. The TEC 332, the heat sink 334, and the fan 330 may be held in place by a housing 336.

Thus, for example, during operation of the cooling element 310, the TEC 332 cools the cooling surface 333 and waste heat warms the warming side. The heat sink 334 conducts waste heat away from the TEC 332. The heat sink 334 transfers waste heat to the surrounding atmosphere via convection. The rate of waste heat transfer from the heat sink 334 to the atmosphere is increased by the fan 330, which creates airflow across the heat sink 334 to exhaust warmed air and deliver cooler air to the heat sink 334.

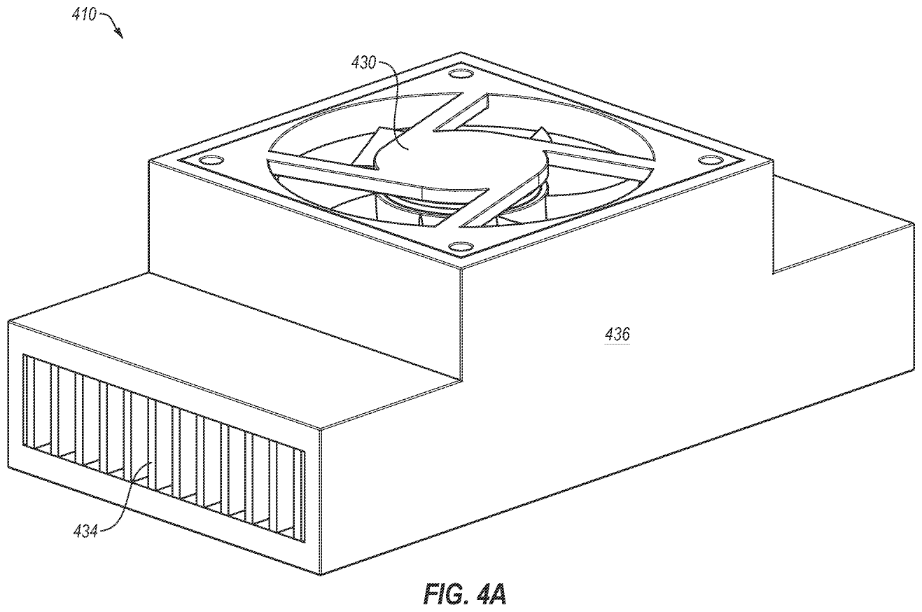

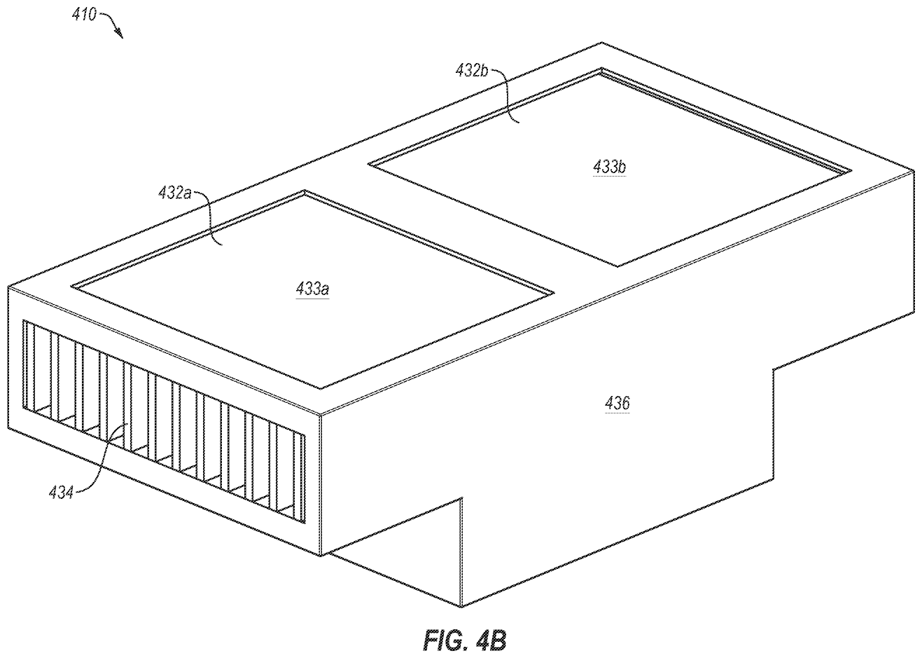

FIGS. 4A and 4B illustrate, respectively, top perspective and bottom perspective views of another example cooling element 410. Electrically conductive lines that supply power to the cooling element 410 are omitted for clarity. The cooling element 410 may generally correspond to the cooling elements 110 of FIG. 1, the cooling elements 210 of FIGS. 2A and 2B, and the cooling element 310 of FIGS. 3A and 3B. The cooling element 410 may include a TEC 432a and a TEC 432b, which may generally correspond to the TEC 332 of FIGS. 3A and 3B. The TEC 432a and the TEC 432b may include, respectively, a cooling surface 433a and a cooling surface 433b, which may generally correspond to the cooling surface 333 of FIGS. 3A and 3B. The cooling element 410 may further include heat sinks 434 positioned on warming surfaces of the TEC 432a and the TEC 432b. The heat sinks 434 may generally correspond to the heat sink 334 of FIGS. 3A and 3B. The cooling element 410 may further include a fan 430, which may generally correspond to the fan 330 of FIGS. 3A and 3B. The TEC 432a, TEC 432b, heat sinks 434, and fan 430 may be held in place by a housing 436.

The cooling element 410 may facilitate cooling via two TECs, the TEC 432a and the TEC 432b, while employing a single fan 430 to draw air across the heat sinks 434. Accordingly, for example, the cooling element 410 may cool more efficiently than a cooling element that employs one fan per TEC, as the cooling element 410 may power half as many fans. In some embodiments, more than two TECs may be associated with a single fan in a cooling element. For example, 3, 4, 5, or more TECs may be associated with a single fan.

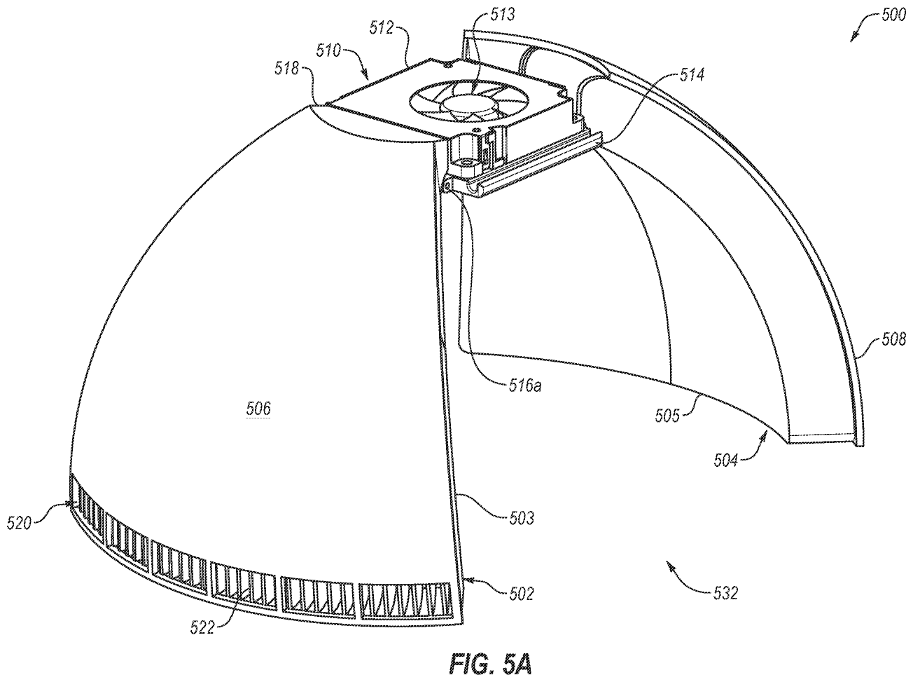

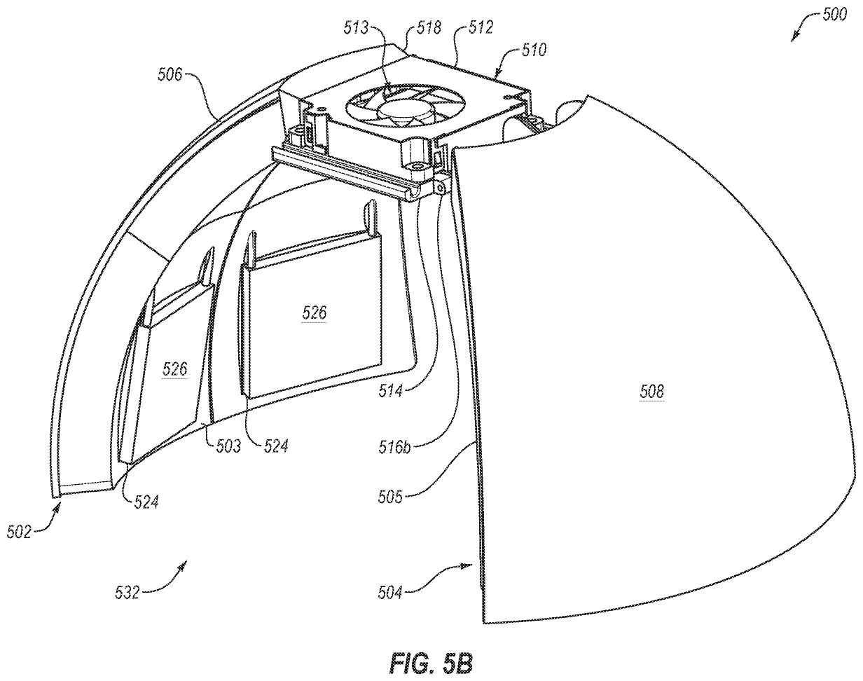

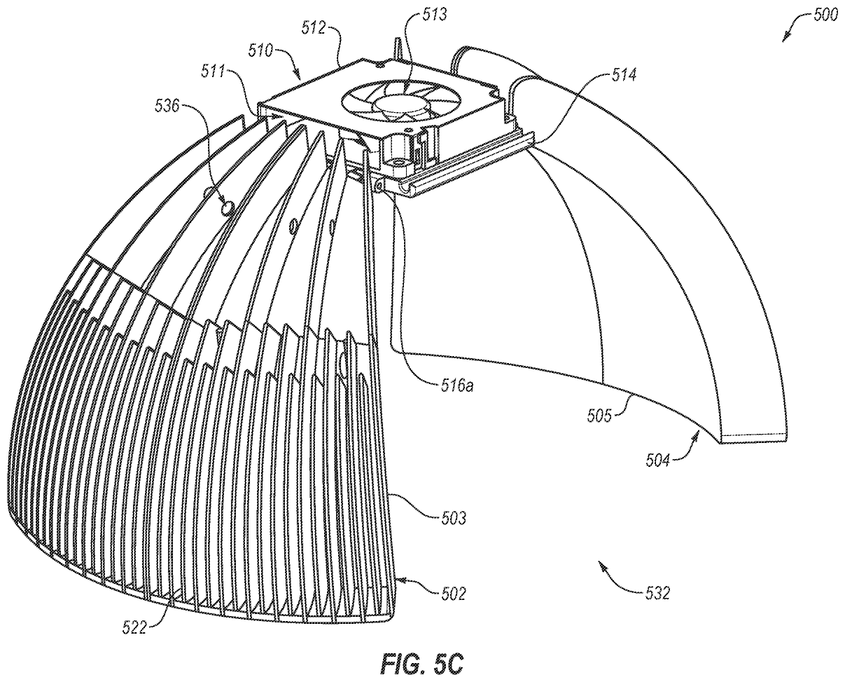

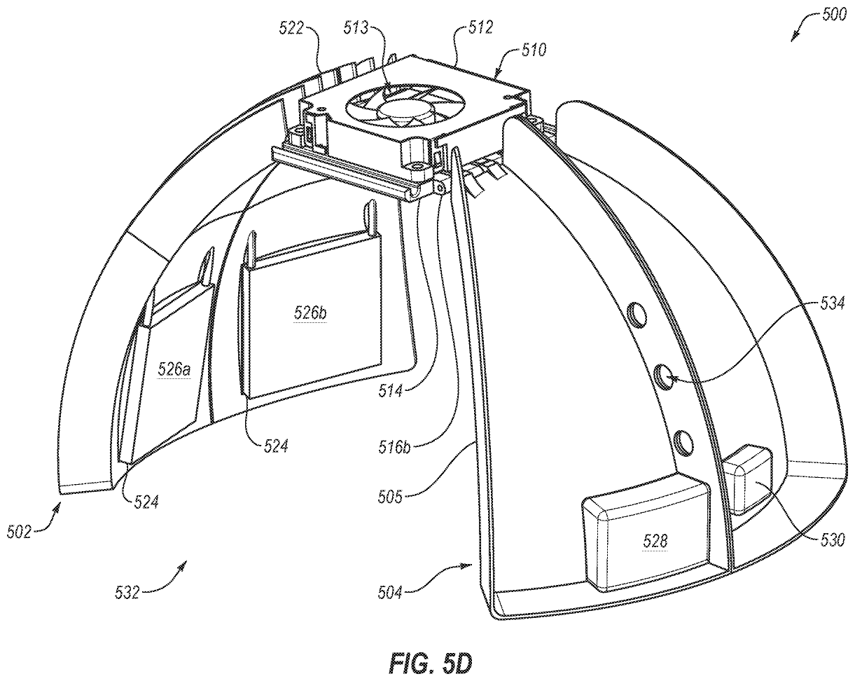

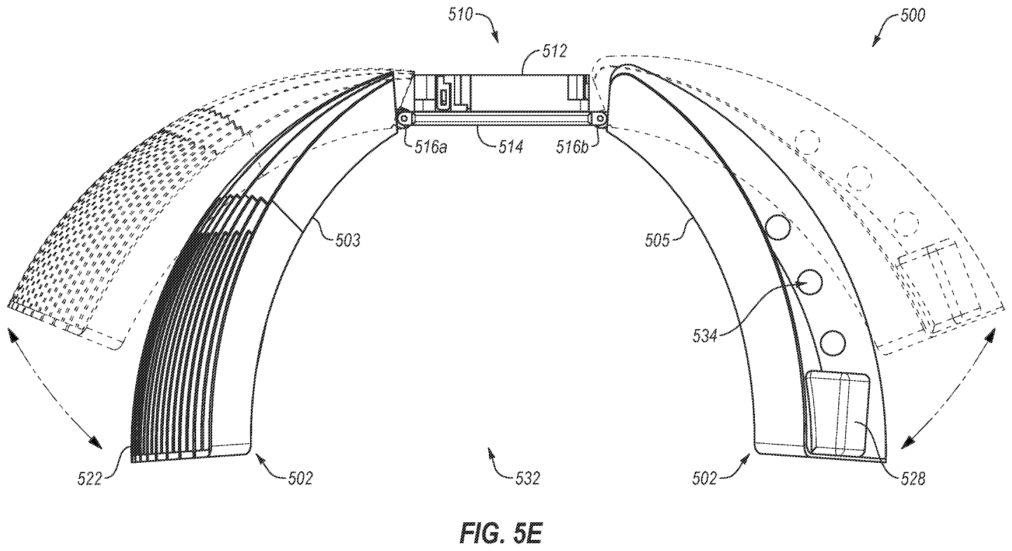

FIGS. 5A and 5B illustrate perspective views of another example headpiece 500, which may generally correspond to the headpiece 100 of FIG. 1 and to the headpiece 200 of FIGS. 2A and 2B. FIGS. 5C and 5D illustrate the headpiece 500 of FIGS. 5A and 5B, respectively, with a cover 506 of a first cap portion 502 and a cover 508 of a second cap portion 504 omitted. FIG. 5E is a front view of the headpiece 500 of FIGS. 5C and 5E.

In some embodiments, the headpiece 500 may include a bridge portion 510 having a base 514 and a fan 512. In some configurations, the base 514 may be a portion of a housing of the fan 512. Alternately, the fan 512 may be separate and fastened to the base 514 via adhesives, threaded fasteners, clips, push-fit connections, and the like.

The base 514 may form a hinge 516a with a body 503 of the first cap portion 502. Alternately or additionally, the base 514 may form a hinge 516b with a body 505 of the second cap portion 504. The first cap portion 502, the second cap portion 504, and the bridge portion 510 together form a shell of the headpiece 500. As may be seen in FIG. 5E, the hinge 516a and/or the hinge 516b may facilitate adjustment of a concavity 532 defined by the first cap portion 502, the second cap portion 504, and the bridge portion 510. The concavity 532 may generally correspond to the concavity 204 of FIG. 2A. An object, such as a human head, which may generally correspond to the head of the user 150 of FIG. 1, may be located within the concavity 532 between the first cap portion 502 and the second cap portion 504. The first cap portion 502 and the second cap portion 504 may be adjected to be generally closer together, which may result in a relatively smaller concavity 532 suitable for a relatively smaller human head. Conversely, the first cap portion 502 and the second cap portion 504 may be adjusted to be generally farther apart, which may result in a relatively larger concavity 532 suitable for a relatively larger human head. A flexible fluid conduit 518 (shown in FIGS. 5A and 5B) may facilitate movement of the first cap portion 502 relative to the bridge portion 510 while facilitating fluid communication between the first cap portion 502 and the fan 512.

The body 503 and the cover 506 may generally form a housing of the first cap portion 502. The body 505 and the cover 508 may generally form a housing of the second cap portion 504.

The headpiece 500 includes at least one TEC 524, which may generally correspond to the TEC 332 of FIG. 3B and the TEC 432a and TEC 432b of FIG. 4B. Although the headpiece 500 is shown to include two TECs 524, in some embodiments the headpiece 500 may include one TEC 524 or more than two TECs 524. Each TEC 524 is positioned on the body 503 of the first cap portion 502 with a cooling surface 526 (shown in FIGS. 5B and 5D) facing the concavity 532 between the first cap portion 502 and the second cap portion 504.

A counterpart heating surface of each TEC 524 may be in thermal communication with a heat sink, such as fins 522 located on the body 503. For example, each TEC 524 may produce waste heat during operation that transfers to the fins 522 by way of conduction. In some embodiments, the fins 522 and at least a portion of the body 503 located between the fins 522 and the TEC 524 may include thermally conductive material such as copper, aluminum, thermally conductive polymers, thermally conductive ceramics, and the like. In some embodiments, a thermal compound may be employed at an interface between each TEC 524 and the body 503 to facilitate the transfer of thermal energy from the TEC 524 to the body 503 and to the fins 522.

The fins 522 may act as a heat exchanger with an environment of the headpiece 500. For example, the fins 522 may facilitate transfer of thermal energy to the environment via convection. In some configurations, the fan 512 may facilitate airflow past the fins 522 to increase a rate of thermal energy transfer from the TEC 524 to the environment. For example, operation of the fan may encourage air from the environment to move through one or more openings 520 in the cover 506, through the space between the cover 506 and the body 503, which moves the air along the fins 522, facilitating thermal energy transfer by providing a supply of relatively cooler air to the fins 522 at an increased rate relative to circulation without a fan. The operation of the fan 512 may further facilitate movement of the air located between the body 503 and cover 506 to move through the flexible fluid conduit 518 (shown in FIGS. 5A and 5B) of the cover 506, through a intake opening 511 of the fan 512 (shown in FIG. 5C) and out through an exhaust opening 513 of the fan 512. In some embodiments, the fan 512 may be omitted and additional openings may be formed in the cover 506 to facilitate thermal energy transfer to the environment via unassisted circulation.

The headpiece 500 may include a battery 528 and a controller 530. The battery 528 may generally correspond to the battery 120 of FIG. 1 and the battery 220 of FIGS. 2A and 2B. The controller 530 may generally correspond to the control module 122 of FIG. 1 and the control module 222 of FIGS. 2A and 2B. In some embodiments, the battery 528 and/or the controller 530 may be located within a housing formed from the body 505 and the cover 508. Positioning the battery 528 and/or the controller 530 with the second cap portion 504 may facilitate a relative balance in wight between the second cap portion 504 and the first cap portion 502, which may facilitate a user wearing the headpiece 500 relatively comfortably. Alternately or additionally, the battery 528 and/or the controller 530 may be located in the first cap portion 502 and/or the bridge portion 510.

Conductive wiring between the battery 528, the controller 530, the fan 512, and each TEC 524 is omitted for clarity. Openings 534 (shown in FIGS. 5D and 5E) in the body 505, openings 536 (shown in FIG. 5C) in the body 503, and channels in the base 514 may facilitate routing of conductive wiring such that power and signaling may be transmitted between each TEC 524, the fan 512, the battery 528, and the controller 530.

In some embodiments, the headpiece 500 may include a liner located on the body 503 of the first cap portion 502, facing the concavity 532. The liner may generally correspond to the first material 206 of FIG. 2A. Alternately or additionally, the headpiece 500 may include a liner located on the cover 508 of the second cap portion 504, facing the concavity 532.

The headpiece 500 may be worn on the head of a user generally corresponding to the user 150 of FIG. 1. For example, the user may be experiencing a migraine, which may be accompanied by allodynia. The user may position the headpiece 500 such that the first cap portion 502 is positioned on the side of the head where the user is experiencing the migraine. The headpiece 500 is configured such that the first cap portion 502 may be positioned on either the right side or the left side of the user's head without alteration of the headpiece 500. Thus, for example, a user experiencing pain and other symptoms associated with a migraine may employ the headpiece 500 simply by orienting the headpiece 500 to locate the first cap portion 502 on the side of the head the user is experiencing the migraine. Furthermore, the user may adjust the positioning of the first cap portion 502 and/or the second cap portion 504 to encourage a comfortable fit and functional contact between each cooling surface 526 and the user's head.

In some configurations, the headpiece 500 may lower the skin temperature of the user's head at each cooling surface 526 by about 10 to 12 degrees Fahrenheit in about 60 seconds of starting operation of the headpiece 500. Experiments show that some configurations may lower a skin temperature of a user at each cooling surface 526 from about 24 degrees Celsius to about 11 degrees Celsius within about 100 seconds of starting operation of the headpiece 500 in an environment that is about 16 degrees Celsius. Experiments further show that some configurations, while the headpiece 500 is not being worn, may lower the temperature of each cooling surface 526 from about 19 degrees Celsius to about 2 degrees Celsius within about 60 seconds of starting operation in an environment that is about 19 degrees Celsius.

The embodiments described in this disclosure may include the use of a special purpose or general-purpose computer including various computer hardware or software modules, as discussed in greater detail below. For example, the control module 122, wireless controller 160, integrated controller 170, control module 222, or controller 530 may include special purpose or general-purpose computer including various computer hardware or software modules.

Embodiments within the scope of this disclosure also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computer” may be any computing system as previously defined herein, or any module or combination of modulates running on a computing system.

For the processes and/or methods disclosed, the functions performed in the processes and methods may be implemented in differing order as may be indicated by context. Furthermore, the outlined steps and operations are only provided as examples, and some of the steps and operations may be optional, combined into fewer steps and operations, or expanded into additional steps and operations.

The present disclosure is not to be limited in terms of the particular embodiments described in this application, which are intended as illustrations of various aspects.

Many modifications and variations can be made without departing from its scope. Functionally equivalent methods and apparatuses within the scope of the disclosure, in addition to those enumerated, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used in this disclosure is for the purpose of describing particular embodiments only, and is not intended to be limiting.

This disclosure may sometimes illustrate different components contained within, or connected with, different other components. Such depicted architectures are merely exemplary, and many other architectures can be implemented which achieve the same or similar functionality.

The terms used in this disclosure, and in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.). In addition, if a specific number of elements is introduced, this may be interpreted to mean at least the recited number, as may be indicated by context (e.g., the bare recitation of “two recitations,” without other modifiers, means at least two recitations, or two or more recitations). As used in this disclosure, any disjunctive word and/or phrase presenting two or more alternative terms should be understood to contemplate the possibilities of including one of the terms, either of the terms, or both terms. For example, the phrase “A or B” will be understood to include the possibilities of “A” or “B” or “A and B.”

Aspects of the present disclosure may be embodied in other forms. The described aspects are to be considered in all respects illustrative and not restrictive. The claimed subject matter is indicated by the appended claims rather than by the foregoing description. All changes which come within the meaning and range of equivalency of the claims are to be embraced within their scope.

What is claimed is:

1. A headpiece comprising:
a shell defining a concavity;
a thermoelectric cooler having a cooling surface and a warming surface;
a heat sink in thermal communication with the warming surface; and
a fan in fluid communication with the heat sink.

2. The headpiece of claim 1, further comprising a plurality of thermoelectric coolers having a plurality of cooling surfaces and a plurality of warming surfaces, the thermoelectric cooler being a first thermoelectric cooler of the plurality of thermoelectric coolers, the warming surface being a first warming surface of the plurality of warming surfaces, and the cooling surface being a first cooling surface of the plurality of cooling surfaces.

3. The headpiece of claim 2, wherein the heat sink is in thermal communication with each warming surface of the plurality of warming surfaces.

4. The headpiece of claim 2, further comprising:
a plurality of heat sinks, the heat sink being a first heat sink of the plurality of heat sinks; and
a plurality of fans, the fan being a first fan of the plurality of fans,
wherein each heat sink of the plurality of heat sinks is in thermal communication with at least one warming surface of the plurality of warming surfaces and each fan of the plurality of fans is in fluid communication with at least one heat sink of the plurality of heat sinks.

5. The headpiece of claim 1, wherein the shell includes:
a first cap portion;
a second cap portion; and
a bridge portion connected to the first cap portion and the second cap portion.

6. The headpiece of claim 5, wherein the bridge portion is connected to the first cap portion by a first hinge.

7. The headpiece of claim 6, wherein the bridge portion is connected to the second cap portion by a second hinge.

8. The headpiece of claim 5, wherein the fan is located on the bridge portion.

9. The headpiece of claim 8, wherein:
the first cap portion includes a first body and a first cover forming a first housing;
the heat sink includes a plurality of thermally conductive fins located within an interior of the first housing; and
the fan is in fluid communication with the interior of the first housing.

10. The headpiece of claim 9, further comprising:
a battery; and
a controller,
wherein the second cap portion includes a second body and a second cover forming a second housing, the battery and the controller located within the second housing.

11. The headpiece of claim 9, further comprising a flexible fluid conduit forming a fluid connection between an intake opening of the fan and the interior of the first housing, wherein the first cover forms an opening adjacent to the thermally conductive fins of the heat sink.

12. A system comprising:
a headpiece including:
a shell defining a concavity;
a thermoelectric cooler having a cooling surface and a warming surface;
a heat sink in thermal communication with the warming surface; and
a fan in fluid communication with the heat sink; and
a mobile device configured to execute an application configured to remotely control operation of the headpiece.

13. The system of claim 12, wherein the application is further configured to collect and store data associated with at least one of:
characteristics of a migraine experienced by a user of the headpiece;
symptoms of the migraine; and
triggering factors associated with the migraine.

14. The system of claim 12, wherein the headpiece further includes a plurality of thermoelectric coolers having a plurality of cooling surfaces and a plurality of warming surfaces, the thermoelectric cooler being a first thermoelectric cooler of the plurality of thermoelectric coolers, the warming surface being a first warming surface of the plurality of warming surfaces, and the cooling surface being a first cooling surface of the plurality of cooling surfaces.

15. The system of claim 14, wherein the shell of the headpiece includes:
a first cap portion;
a second cap portion; and
a bridge portion connected to the first cap portion and the second cap portion.

16. The system of claim 15, wherein the bridge portion is connected to the first cap portion by a first hinge and the bridge portion is connected to the second cap portion by a second hinge.

17. The system of claim 16, wherein the fan is located on the bridge portion.

18. The system of claim 17, wherein:
the first cap portion includes a first body and a first cover forming a first housing;
the heat sink includes a plurality of thermally conductive fins located within an interior of the first housing; and
the fan is in fluid communication with the interior of the first housing.

19. The system of claim 18, the headpiece further including:
a battery; and
a controller,
wherein the second cap portion includes a second body and a second cover forming a second housing, the battery and the controller located within the second housing.

20. The system of claim 18, the headpiece further including a flexible fluid conduit forming a fluid connection between an intake opening of the fan with the interior of the first housing, wherein the first cover forms an opening adjacent to the thermally conductive fins of the heat sink.
